(19) United States
(12) Patent Application Publication
(10) Pub. No.: US 2025/0128087 A1
(43) Pub. Date: Apr. 24, 2025

(54) MASSAGING DEVICE

(71) Applicant: SHENZHEN KAIYAN MEDICAL EQUIPMENT CO, LTD., Shenshen (CN)

(72) Inventors: ALAIN DIJKSTRA, Amstelveen (NL); LI XIANG, Shenzhen (CN)

(73) Assignee: SHENZHEN KAIYAN MEDICAL EQUIPMENT CO., LTD, Shenzhen (CN)

(21) Appl. No.: 18/533,237

(22) Filed: Dec. 8, 2023

(30) Foreign Application Priority Data
Oct. 23, 2023 (CN) 2023228414638

(51) Int. Cl. A61N 5/06; A61H 23/02; A61N 1/36; H02J 50/10

(52) U.S. Cl. CPC A61N 5/0616; A61H 23/02; H02J 50/10; A61H 2201/10; A61H 2201/169; A61N 1/36014; A61N 2005/0626; A61N 2005/0652; A61N 2005/0659; A61N 2005/0663

(57) ABSTRACT

Embodiments of the present invention provide a massaging device. The massaging device includes an enclosing structure defining a cavity. The enclosing structure includes a plurality of lateral portions, a top portion and a bottom portion. The massaging device further includes a plurality of Light Emitting Diodes (LEDs) enclosed within the cavity. Also, the massaging device includes a plurality of diaphanous portions provided partially in the plurality of lateral portions, partially in the top portion, and partially in the bottom portion.

### TECHNICAL FIELD

The present invention generally relates to therapeutic devices. More specifically the present invention relates to light or electromagnetic radiation-based therapeutic devices.

### BACKGROUND ART

Traditionally, massaging stones have been used for skin rejuvenation. Such stones scrape the skin to induce redness in the skin and then the skin heals itself to obtain rejuvenated skin. For example, US20140213944A1 discloses a hand-held massage device and methods for applying pressure therapies to various parts of the anatomy using the device. The device is an elongated, rigid, substantially planar member, configured with a series of alternating concave and convex shapes, and a knob-like projecting member at an end thereof. However, such devices are rudimentary in construction and provide a very minimal therapeutic effect.

Therefore, there is a need for a device that overcomes the disadvantages and limitations associated with the prior art and provides a more satisfactory solution.

### OBJECTS OF THE INVENTION

Some of the objects of the invention are as follows:

An object of the invention is to provide a massaging device for providing skin rejuvenation through light therapy from several Light Emitting Diodes (LEDs).

Another object of the invention is to provide a massaging device with diaphanous portions such that the LEDs are covered by the diaphanous portions.

Another object of the invention is to provide a massaging device where a control unit can communicate with an external communication device.

Yet another object of the invention is to provide a massaging device that can be charged wirelessly.

### SUMMARY OF THE INVENTION

According to a first aspect of the present invention, there is provided a massaging device. The massaging device includes an enclosing structure defining a cavity. The enclosing structure includes a plurality of lateral portions, a top portion and a bottom portion. The massaging device further includes a plurality of Light Emitting Diodes (LEDs) enclosed within the cavity. Also, the massaging device includes a plurality of diaphanous portions. The plurality of diaphanous portions are provided partially in the plurality of lateral portions, partially in the top portion, and partially in the bottom portion.

In one embodiment of the invention, the plurality of lateral portions of the enclosing structure include at least two capacitive touch switches, such that, the control unit is configured to be activated on receiving touch inputs from the at least two capacitive touch switches

In one embodiment of the invention, the plurality of diaphanous portions include a plurality of crest-shaped segments and a plurality of trough-shaped segments, and the plurality of LEDs are located in line with the plurality of trough-shaped segments.

In one embodiment of the invention, the plurality of LEDs is configured to emit radiation in red and infrared regions of the electromagnetic spectrum.

In one embodiment of the invention, the massaging device further includes a battery configured to be charged wirelessly using a wireless charging coil.

In one embodiment of the invention, the massaging device further comprises a wireless charger including an induction coil and a cavity acting as a seat for docking of the enclosing structure onto the wireless charger.

In one embodiment of the invention, the wireless charger is configured to be powered through a USB interface.

In one embodiment of the invention, the massaging device, further includes a control unit configured to control the irradiation characteristics of the plurality of LEDs.

In one embodiment of the invention, the control unit is enclosed by the enclosing structure.

In one embodiment of the invention, the irradiation characteristics include a mode of operation of the plurality of LEDs and frequencies of the irradiation.

In one embodiment of the invention, the control unit includes a communication interface configured to connect the massaging device with an external communication device and the control unit is configured to receive a control input from the external communication device.

In one embodiment of the invention, the control input includes a signal for modification of irradiation characteristics of the plurality of LEDs.

In one embodiment of the invention, the enclosing structure is made up of a material selected from a group consisting of Acrylonitrile Butadiene Styrene (ABS), Polycarbonate (PC), natural stone, Himalayan stone, and combinations thereof.

In one embodiment of the invention, the massaging device further comprises a vibratory motor configured to provide vibratory massage to the body of a user.

In one embodiment of the invention, the massaging device further comprises a plurality of electrodes configured to provide Transcutaneous Electrical Nerve Stimulation (TENS), Electronic Muscle Stimulation (EMS), and Microcurrent Electrical Therapy (MET) to the body of a user.

In one embodiment of the invention, the enclosing structure is formed in shape of a Gua Sha stone.

According to a second aspect of the present invention, there is provided a massaging device. The massaging device includes an enclosing structure defining a cavity. The enclosing structure includes a plurality of lateral portions, a top portion and a bottom portion. The massaging device further includes a plurality of Light Emitting Diodes (LEDs) enclosed within the cavity. Also, the massaging device includes a plurality of diaphanous portions. The plurality of diaphanous portions are provided partially in the plurality of lateral portions, partially in the top portion, and partially in the bottom portion. The plurality of diaphanous portions includes a plurality of crest-shaped segments and a plurality of trough-shaped segments, and the plurality of LEDs are located in line with the plurality of trough-shaped segments.

According to a third aspect of the present invention, there is provided a method of using a massaging device. The method includes activating the massaging device using a user interface. Furthermore, the method includes providing a control input to a control unit of the massaging device, from one or more of the user interface and an external computing device. The method also includes applying a plurality of diaphanous portions to a face of a user.

In one embodiment of the invention, the method further includes activating a vibratory motor of the massaging device and providing a vibratory massage to the body of the user.

In one embodiment of the invention, the method further includes providing one or more of Transcutaneous Electrical Nerve Stimulation (TENS), Electronic Muscle Stimulation (EMS), and Microcurrent Electrical Therapy (MET) to a body of the user using a plurality of electrodes of the massaging device.

In the context of the specification, the terms “massage” or “massaging” refers to manipulation of tissues (as by pressing, rubbing, kneading, or tapping) with the hand or an instrument for relaxation or therapeutic purposes. The instrument in that regard may have a relatively smooth surface or an abrasive surface.

In the context of the specification, the term “rubbing” refers to an act of moving along a surface of the body with pressure.

In the context of the specification, the term “pressing” refers to acting upon through steady pushing or thrusting force exerted in contact.

In the context of the specification, the term “processor” refers to one or more of microprocessors, a microcontroller, a general-purpose processor, a Field Programmable Gate Array (FPGA) or an Application Specific Integrated Circuit (ASIC), and the like.

In the context of the specification, the phrase “memory unit” refers to volatile storage memory, such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc.

In the context of the specification, the phrase “storage device” refers to a non-volatile storage memory such as EPROM, EEPROM, flash memory, or the like.

In the context of the specification, the phrase “communication interface” refers to a device or a module enabling direct connectivity via wires and connectors such as USB, HDMI, VGA, or wireless connectivity such as Bluetooth or Wi-Fi or Local Area Network (LAN) or Wide Area Network (WAN) implemented through TCP/IP, IEEE 802.x, GSM, CDMA, LTE or other equivalent protocols.

In the context of the specification, the term “historical” in execution of a command refers to anything pertaining to a time instant(s) that is earlier than a time instant of an initiation of the command.

In the context of the specification, the term, “real-time”, refers to without intentional delay, given the processing limitations of hardware/software/firmware involved and the time required to accurately measure/receive/process/transmit data as practically possible.

In the context of the specification, the phrase “diaphanous” refers to a material that allows electromagnetic radiation to pass through them, either partially or in entirety. In that regard, diaphanous materials are envisaged to include both transparent and translucent materials.

In the context of the specification, the phrase “Gua Sha stone” refers to a stone used in ancient Chinese medicine for scraping the skin of the face to promote blood circulation, produce collagen, decrease inflammation, and for providing other known health benefits.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelength, wherein the range is inclusive of UV and IR frequencies and wavelengths. It is to be noted here that UV radiation can be categorized in several manners depending on respective wavelength ranges, all of which are envisaged to be under the scope of this invention. For example, UV radiation can be categorized as, Hydrogen Lyman-? (122-121 nm), Far UV (200-122 nm), Middle UV (300-200 nm), Near UV (400-300 nm). The UV radiation may also be categorized as UVA (400-315 nm), UVB (315-280 nm), and UVC (280-100 nm) Similarly, IR radiation may also be categorized into several categories according to respective wavelength ranges which are again envisaged to be within the scope of this invention. A commonly used subdivision scheme for IR radiation includes Near IR (0.75-1.4 ?m), Short-Wavelength IR (1.4-3 ?m), Mid-Wavelength IR (3-8 ?m), Long-Wavelength IR (8-15 ?m) and Far IR (15-1000 ?m).

In the context of the specification, a “polymer” is a material made up of long chains of organic molecules (having eight or more organic molecules) including, but not limited to, carbon, nitrogen, oxygen, and hydrogen as their constituent elements. The term polymer is envisaged to include both naturally occurring polymers such as wool, and synthetic polymers such as polyethylene and nylon.

In the context of the specification, “Light Emitting Diodes (LEDs)” refer to semiconductor diodes capable of emitting electromagnetic radiation when supplied with an electric current. The LED are characterized by their superior power efficiencies, smaller sizes, rapidity in switching, physical robustness, and longevity when compared with incandescent or fluorescent lamps. In that regard, the plurality of LEDs may be through-hole type LEDs (generally used to produce electromagnetic radiations of red, green, yellow, blue and white colors), Surface Mount Technology (SMT) LEDs, Bi-color LEDs, Pulse Width Modulated RGB (Red-Green-Blue) LEDs, and high-power LEDs, etc.

Materials used in the one or more LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, and Boron, and Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used for generating a wider color gamut. White and other colored lightings may also be produced using phosphor coatings such as Yttrium Aluminum Garnet (YAG) in combination with a blue LED to generate white light and Magnesium doped potassium fluorosilicate in combination with blue LED to generate red light. Additionally, near Ultraviolet (UV) LEDs may be combined with europium-based phosphors to generate red and blue lights and copper and zinc doped zinc sulfide-based phosphor to generate green light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, discussion on generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, the one or more LEDs may also be micro-LEDs described through U.S. Pat. Nos. 8,809,126 B2, 8,846,457 B2, 8,852,467 B2, 8,415,879 B2, 8,877,101 B2, 9,018,833 B2 and their respective family members, assigned to NthDegree Technologies Worldwide Inc., which are included herein by reference, in their entirety. The one or more LEDs, in that regard, may be provided as a printable composition of the micro-LEDs, printed on a substrate.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1A illustrates a perspective view of the massaging device, in accordance with an embodiment of the present invention;

FIG. 1B illustrates an exploded view of the massaging device, in accordance with an embodiment of the present invention;

FIG. 1C illustrates an exploded view of the massaging device, in accordance with another embodiment of the present invention;

FIG. 1D illustrates a perspective view of the massaging device in accordance with another embodiment of the invention;

FIG. 2 illustrates an exploded view of the massaging device being charged wirelessly, in accordance with an embodiment

FIG. 3 illustrates an exploded view of the massaging device illustrating a control unit and a wireless charger, in accordance with an embodiment of the present invention;

FIG. 4 illustrates the massaging device connected with an external communication device, in accordance with an embodiment of the present invention;

FIG. 5 illustrates a user using the massaging device, in accordance with an embodiment of the present invention; and

FIG. 6 illustrates a method of using the massaging device, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

Embodiments of the present invention disclose a massaging device that provides skin rejuvenation using light therapy. The massaging device has been shaped in form of a Gua Sha stone with an enclosing structure including several diaphanous portions. The diaphanous portions may be provided in top, bottom, and lateral portions of the enclosing structure. The diaphanous portions encapsulate several Light Emitting Diodes (LEDs) that provide light therapy in red and infrared frequencies. A control unit controls the LEDs. The massaging device is also envisaged to be charged wirelessly. Referring to the drawings, the invention will now be explained in further detail.

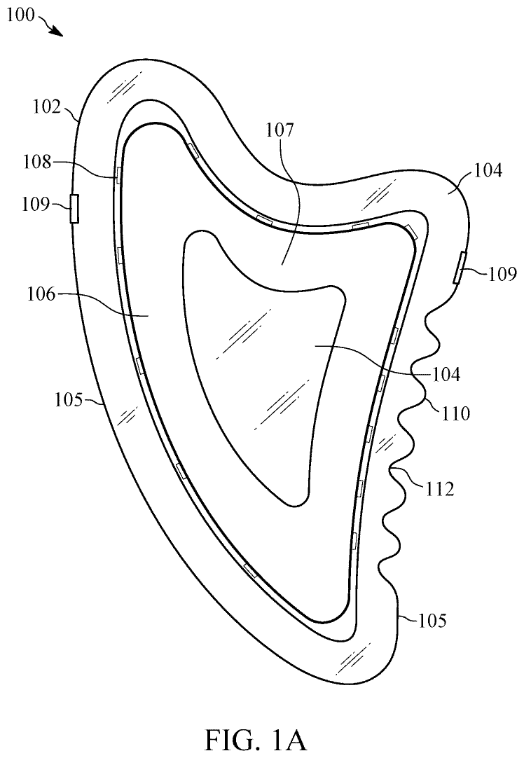

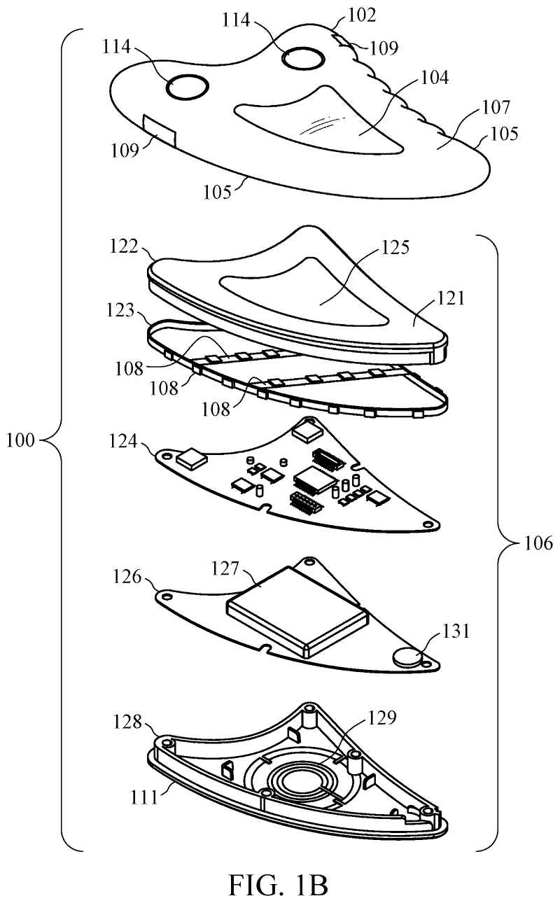

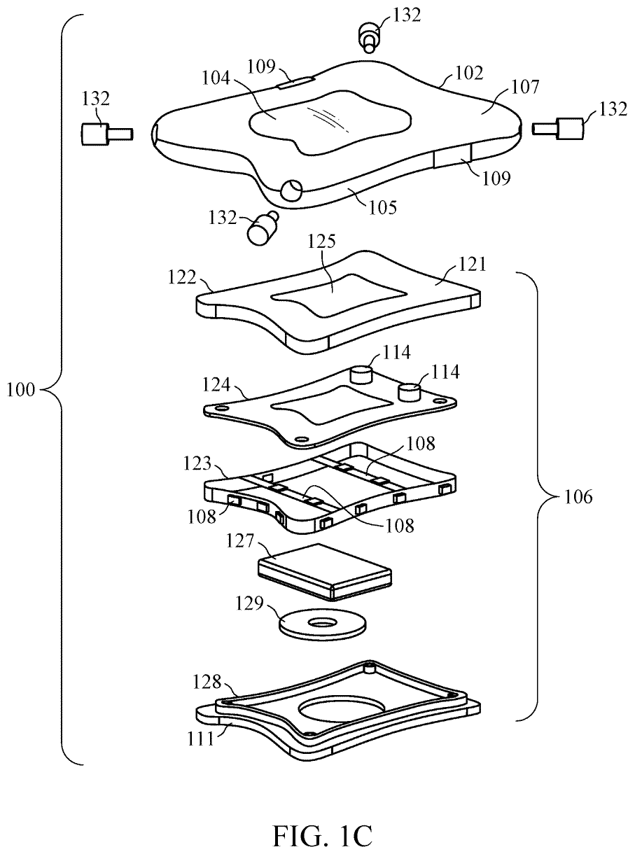

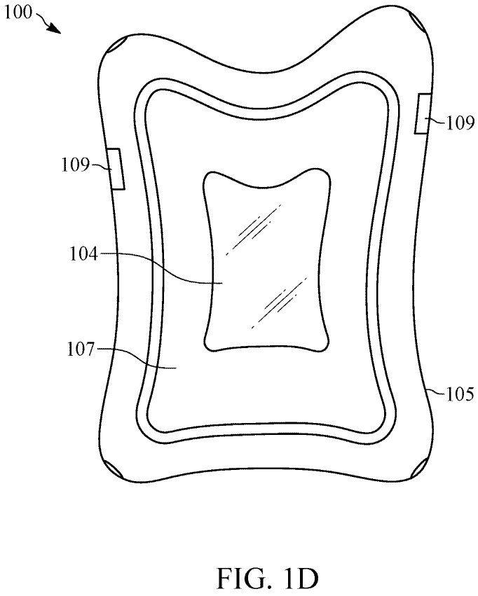

FIG. 1A illustrates a perspective view of the massaging device 100 (hereinafter referred to as “the device 100”), in accordance with an embodiment of the present invention. The device 100 includes an enclosing structure 102. In several embodiments of the invention, the enclosing structure 102 is made up of a polymeric material and is shaped in form of a stone. In several embodiments of the invention, the enclosing structure 102 is shaped in form of a Gua Sha stone. In several embodiments of the invention, the enclosing structure 102 is made up of Acrylonitrile Butadiene Styrene (ABS) material. The ABS material is known for its high impact resistance, high rigidity, heat and chemical resistance, and structural and dimensional stability. However, in several alternate embodiments, ABS may be combined with Polycarbonate (PC) material. In several embodiments of the invention, the enclosing structure 102 is made up of natural or Himalayan stone. Thus, the device 100 has been adapted to resemble a stone.

Further, the enclosing structure 102 includes a plurality of diaphanous portions 104. The plurality of diaphanous portions 104 may be made from combinations of several polymeric materials used to make transparent objects. For example, the plurality of diaphanous portions 104 may be made from any one or more of Polycarbonate, PMMA or Acrylic, Polyethylene Terephthalate (PET), Amorphous Co-polyester (PETG), Polyvinyl Chloride (PVC), Liquid Silicone Rubber (LSR), Cyclic Olefin Copolymers, Polyethylene (PE), Polystyrene (PS), Thermoplastic polyurethanes (TPU), Polyvinyl butyral (PVB), Co-polymer ethylene vinyl acetate (EVA). The plurality of diaphanous portions 104 may partially be provided in a plurality of lateral portions 105 of the enclosing structure 102. Further, the plurality of diaphanous portions 104 may partially be provided in a top portion 107 of the enclosing structure 102. Moreover, the plurality of diaphanous portions 104 may partially be present in a bottom portion 111 (See FIG. 1B, 1C, 3) of the enclosing structure 102. The plurality of diaphanous portions 104 may be integrally formed with the rest of the enclosing structure 102 or may be attached to the plurality of lateral portions 105, the top portion 107, or the bottom portion 111 through fastening means such as snap-fit arrangements, adhesive bonding, fasteners such as screws, bolts, and rivets, etc.

The enclosing structure 102 due to its shape encloses a cavity. The cavity includes a plurality of LEDs 108. The plurality of LEDs 108 is included in the cavity in a manner that the plurality of LEDs 108 is enclosed by the plurality of diaphanous portions 104 of the enclosing structure 102. This allows the irradiation from the plurality of LEDs 108 to emitted outwards of the enclosing structure 102. It is to be noted here that, in several embodiments of the invention, the plurality of LEDs 108 is configured to emit radiation in red and infrared regions of the electromagnetic spectrum. Moreover, the plurality of diaphanous portions 104 includes a plurality of crest-shaped segments 110 and a plurality of trough-shaped segments 112, and the plurality of LEDs 108 are located in line with the plurality of trough-shaped segments 112. The device 100 also includes a control unit 106 configured to control the irradiation characteristics of the plurality of LEDs 108. Further, in several embodiment of the invention, the plurality of lateral portions 105 of the enclosing structure 102 includes at least two capacitive touch switches 109. The control unit 106 may be configured to be activated on receiving touch inputs from the at least two capacitive touch switches 109.

FIG. 1B illustrates an exploded view of the massaging device 100, in accordance with an embodiment of the present invention. As illustrated in FIG. 1B, the enclosing structure 102 is formed by coupling the top portion 107 and the bottom portion 111 and including several other components between the top portion 107 and the bottom portion 111. The top portion 107 and the bottom portion 111 may be coupled using removable fasteners such as screws or may be coupled using snap fit arrangements to ensure that the top portion 107 and the bottom portion 111 are easily decoupled for repair and/or reconstruction of the massaging device 100 or for servicing such as replacing a battery 127. The massaging device 100 also includes a vibratory motor 131 configured to provide vibratory massage to the body of a user. The vibratory motor 131 may be any one of the types: Eccentric Rotating Mass (ERM), Linear Resonant Actuators (LRA), or Solenoid Actuators.

Also, FIG. 1B illustrates the massaging device 100 including an upper hollow body 122 and a lower hollow body 128. The upper hollow body 122 and the lower hollow body 128 are provided between the top portion 107 and the bottom portion 111 and combine with each other to form a container containing almost all of the electronic components of the massaging device 100. The upper hollow body 122 and the lower hollow body 128 may therefore be provided with several features for Ingress Protection (IP), the features including sealing materials such as a sealant, sealing rings, gaskets, dust and water-resistant coatings, etc. It is envisaged that the upper hollow body 122 includes a part of the plurality of diaphanous portions 125 in a top hollow body portion 121 of the upper hollow body 122. FIG. 1B further illustrates an LED mounting bracket 123 on which the plurality of LEDs 108 has been mounted. In several embodiments of the invention, the plurality of LEDs 108 is mounted on the perimeter of the LED mounting bracket 123, using Surface Mounting Technology (SMT) or through-hole technology. In several alternate embodiments of the invention, the plurality of LEDs 108 may be mounted on the perimeter of the LED mounting bracket 123 and also in a cross direction across the LED mounting bracket 123, such that the plurality of LEDs 108 pass from under the plurality of diaphanous portions 125 and the top portion 107. In that regard, the irradiation from the plurality of LEDs 108 may also be received from central portions of the massaging device 100.

In between the upper 122 and the lower 128 hollow bodies, are located a Printed Circuit Board (PCB) 124, and a battery board 126. The battery board 126 includes the battery 127. In several embodiments of the invention, the battery 127 is a rechargeable battery of Lithium-ion, Lithium-polymer, or Nickel Metal Hydride type. The lower hollow body 128 further includes a wireless charging coil 129 configured to receive power wirelessly from an induction coil of a wireless charger. In that regard, the battery board 126 may also be used to mount the vibratory motor 131. Furthermore, conducting connections may be provided between the battery 127 and the PCB 124, so that electrical power from the battery 127 may be transmitted to other electronic components of the massaging device 100 via the PCB 124. Moreover, conducting connections may also be provided between the PCB 124 and the vibratory motor 131 so that the vibratory motor 131 may be actuated whenever a corresponding signal is received from a user interface 114 provided in the enclosing structure 102.

The user interface 114 may include on/off switches, mode selector switches, play and pause buttons, a touch-based interface, and the like. The user interface 114 has been provided flush with the top portion 117 in counterbore or countersink holes so that the user interface 114 is not damaged while massaging device 100 is being acted upon the body of a user. Moreover, it is envisaged that the upper hollow body 122 is made up of a flexible material so that any pressure applied on to the user interface 114 is transferred to corresponding pressure sensitive portions of the PCB 124. Alternately, a conducting connection may be provided between the user interface 114 and the PCB 124, such that conducting connection either bypasses the upper hollow body 122 or runs through a hole or a slot provided in the upper hollow body 122.

FIG. 1C illustrates an exploded view of the massaging device 100, in accordance with another embodiment of the present invention. FIG. 1D illustrates a perspective view of the massaging device in accordance with another embodiment of the invention. FIG. 1C further illustrates another embodiment of the LED mounting bracket 123 on which the plurality of LEDs 108 has been mounted. In several embodiments of the invention, the plurality of LEDs 108 is mounted on the perimeter of the LED mounting bracket 123, using Surface Mounting Technology (SMT) or through-hole technology. In several alternate embodiments of the invention, the plurality of LEDs 108 may be mounted on the perimeter of the LED mounting bracket 123 and also in the cross direction across the LED mounting bracket 123, such that the plurality of LEDs 108 pass from under the plurality of diaphanous portions 125 of the upper hollow body 122 and the top portion 107 of the enclosing structure 102. In that regard, the irradiation from the plurality of LEDs 108 may also be received from central portions of the embodiments of the massaging device 100 illustrated in FIG. 1C.

FIG. 1C also illustrates a plurality of electrodes 132 provided along the enclosing structure 102. The plurality of electrodes 132 may be provided at edges and/or corners of the enclosing structure 102, the top portion 107, or the bottom portion 111 by threaded fastening, snap-fitting using spring loaded levers in the plurality of electrodes 132. Alternately, the plurality of electrodes 132 may be press-fitted into holes provided at the edges and/or corners of the enclosing structure 102, the top portion 107, or the bottom portion 111. The plurality of electrodes 132 is configured to provide Transcutaneous Electrical Nerve Stimulation (TENS), Electronic Muscle Stimulation (EMS), and Microcurrent Electrical Therapy (MET) to the body of a user.

TENS therapy uses low-voltage currents to provide pain relief. Electrical impulses are delivered through electrodes placed on the surface of the body of the user. The electrodes are placed at or near nerves where the pain is located or at certain known trigger points. EMS therapy is similar to TENS therapy, the difference being that EMS is applied to key muscle groups instead of a generalized application. The electrical signals in EMS cause certain muscles to undergo contractions and tightening. Moreover, electrical impulses in EMS are stronger when compared with TENS therapy. MET in contrast uses a current of amplitude less than 1 milliampere and a frequency of 0.5 Hz and is indicated for the treatment of pain.

In that manner, the plurality of electrodes 132 may be used to provide a current of varying frequencies and amplitude to choose between TENS, EMS and MET. The plurality of electrodes 132 in that regard may be powered using the battery 127 and the flow of current to the plurality of electrodes 132 may be controlled by the control unit 106. For example, very small amperage current (in the order of milli-Amperes and micro-Amperes) may be delivered to the plurality of electrodes 132 for providing the MET. Similarly, respective current values rated for TENS and EMS may be delivered to the plurality of electrodes 132 by the control unit 106 as and when respective therapies are selected to be performed by the user. The user may switch between several therapies such as TENS, EMS, and MET using a mode selector switch of the user interface 114. As a safety feature, the massaging device 100 has been provided with a leakage current detection circuit (not shown) configured to detect flow of leakage current and the control unit 106 is configured to break the flow of the current in the circuitry of the massaging device 100 on detection of the leakage current.

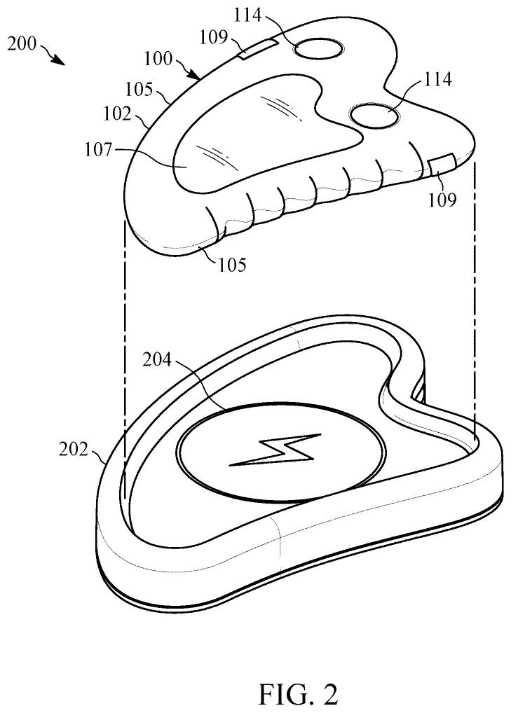

FIG. 2 illustrates an exploded view of the device 100 being charged wirelessly, in accordance with an embodiment 200 of the present invention. The device 100 includes a wireless charger 202 including a cavity acting as a seat for the docking of the enclosing structure 102 onto the wireless charger 202. In that regard, the device 100 includes the rechargeable battery 127 of the type selected from Lithium-ion, Lithium polymer, Nickel metal hydride, and the like. The wireless charger 202 includes an induction coil 204. The induction coil 204 may generate a magnetic field and generate an electromagnetic coupling with the wireless charging coil 129 of the control unit 106 to induce a charging current in the wireless charging coil 129. The charging current would then charge the battery 127.

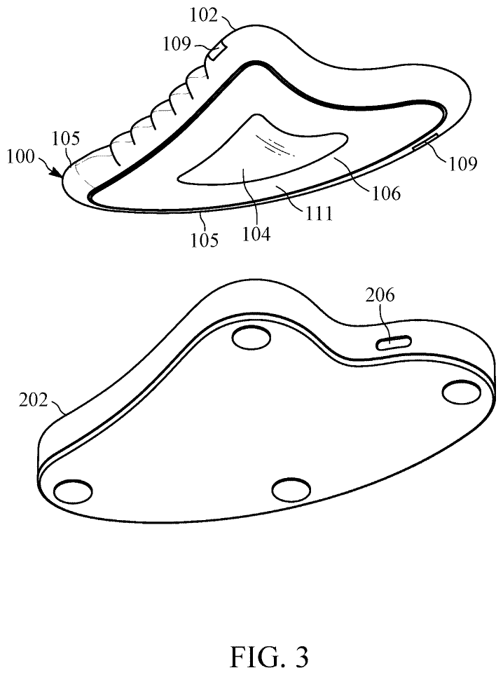

FIG. 3 illustrates an exploded view of the device 100 illustrating the control unit 106 and the wireless charger 202, in accordance with an embodiment of the present invention. In several embodiments of the invention, the control unit 106 includes a processor and a memory unit. The memory unit includes machine-readable instructions for the processor which are executed by the processor for controlling the irradiation characteristics of the plurality of LEDs 108. For example, the processor executing the machine-readable instructions may operate the plurality of LEDs 108 in pulsed or continuous mode or may modify the frequencies of the plurality of LEDs 108. Further, the wireless charger 202 is configured to be powered through a USB interface 206. The control unit 106 also includes a communication interface.

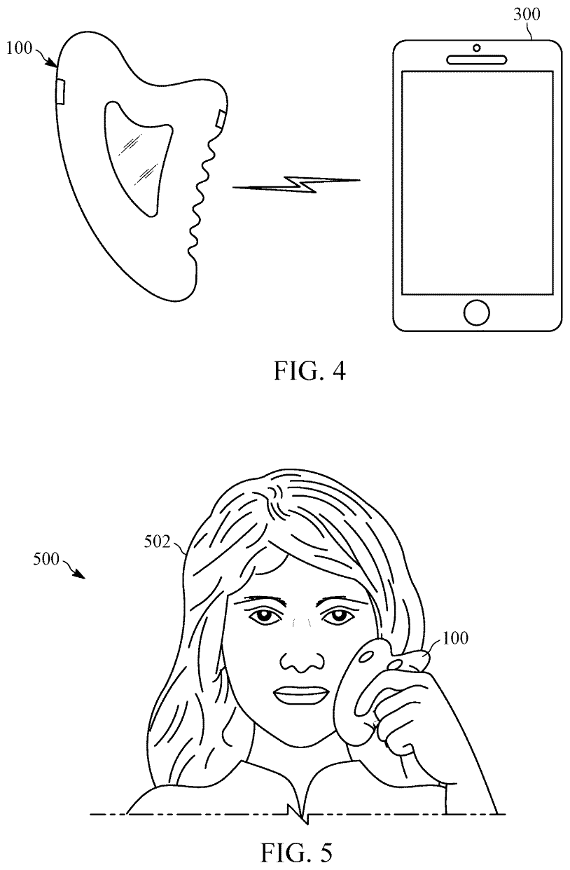

FIG. 4 illustrates the massaging device 100 connected with an external communication device 300, in accordance with an embodiment of the present invention. The communication interface allows the device 100 to connect with the external communication device 300. The external communication device 300 may be a smartphone, a tablet PC, a notebook PC, a desktop, or the like. Further, the control unit 106 is capable of receiving a control input from the external communication device 300 through the communication interface. The control input may include a signal for modifying the irradiation characteristics of the plurality of LEDs 108.

In that manner, the control input may change the operation of the plurality of LEDs 108 from pulse mode to continuous mode or vice versa. Alternately, the control input may be a signal for modifying the frequencies of the irradiation emitted by the plurality of LEDs 108. In several embodiments of the invention, the external communication device 300 may be configured to display the status of the device 100. In that manner, the external communication device 300 may display the ON/OFF status of the device 100 or may display the mode of operation and frequency of irradiation of the plurality of LEDs 108.

FIG. 5 illustrates a user 502 using the device 100, in accordance with an embodiment 500 of the present invention. In use, the user 502 will need to switch on the device 100 from the user interface 114 and apply the device 100 on the skin of the user. The radiation emitted by the plurality of LEDs 108 will cause skin rejuvenation and renewal of the skin of the user 502. Moreover, the vibratory motor 131 will provide vibratory massage to the body of the user 502. Also, in several embodiments of the invention, the user 502 may use the mode selector switch of the user interface 114 to select between TENS, EMS, and MET therapy received through the plurality of electrodes 132.

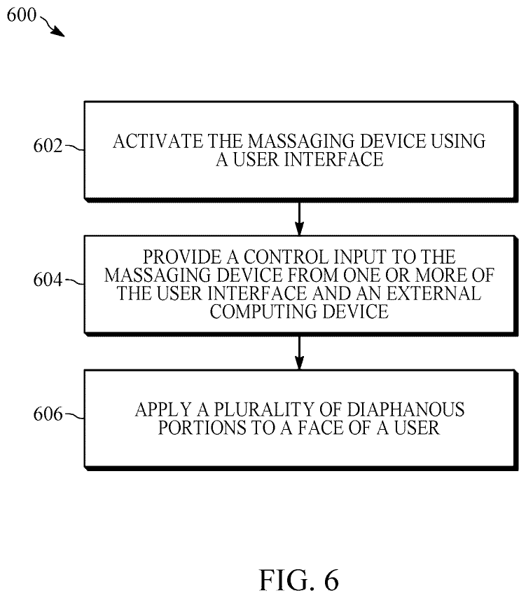

FIG. 6 illustrates a method 600 of using the massaging device 100, in accordance with an embodiment of the present invention. The method 600 begins at Step 602 when the massaging device 100 is activated using the user interface 114. Once the massaging device 100 is activated, the control unit 106 is also activated. At Step 604, the user 502 provides the control input to the control unit 106. The control input may be provided using the user interface 114, the external communication device 300, or a combination of the user interface 114 and the external communication device 300. On receiving the control input, the control unit 106 activates the plurality of LEDs 108. The control input may change the operation of the plurality of LEDs 108 from pulse mode to continuous mode or vice versa. Alternately, the control input may be a signal for modifying the frequencies of the irradiation emitted by the plurality of LEDs 108. At Step 606, the user 502 applies the plurality of diaphanous portions 104 to their face to provide therapeutic and recreational effects. In several embodiments of the invention, the method 600 further includes activating the vibratory motor 131 of the massaging device 100 and providing a vibratory massage to the body of the user 502. Furthermore, in several embodiments of the invention, providing one or more of the TENS, EMS, and MET to the body of the user 502 using the plurality of electrodes 132 of the massaging device 100.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to be providing the broadest scope consistent with the principles and the novel and inventive features disclosed or suggested herein. Accordingly, the invention is anticipated to hold on to all other such alternatives, modifications, and variations that fall within the scope of the present invention and appended claims.

1. A massaging device, the massaging device comprising:
an enclosing structure defining a cavity, the enclosing structure comprising a plurality of lateral portions, a top portion and a bottom portion;
a plurality of Light Emitting Diodes (LEDs) enclosed within the cavity; and
a plurality of diaphanous portions, the plurality of diaphanous portions provided partially in the plurality of lateral portions, partially in the top portion, and partially in the bottom portion.

2. The massaging device as claimed in claim 1, wherein the plurality of lateral portions of the enclosing structure comprises at least two capacitive touch switches, such that, the control unit is configured to be activated on receiving touch inputs from the at least two capacitive touch switches.

3. The massaging device as claimed in claim 1, wherein the plurality of diaphanous portions comprises a plurality of crest-shaped segments and a plurality of trough-shaped segments, and the plurality of LEDs are located in line with the plurality of trough-shaped segments.

4. The massaging device as claimed in claim 1, wherein the plurality of LEDs is configured to emit radiation in red and infrared regions of the electromagnetic spectrum.

5. The massaging device as claimed in claim 1, further comprising a battery configured to be charged wirelessly using a wireless charging coil.

6. The massaging device as claimed in claim 1, further comprising a wireless charger comprising an induction coil and a cavity acting as a seat for docking of the enclosing structure onto the wireless charger.

7. The massaging device as claimed in claim 6, wherein the wireless charger is configured to be powered through a USB interface.

8. The massaging device as claimed in claim 1, further comprising a control unit configured to control the irradiation characteristics of the plurality of LEDs.

9. The massaging device as claimed in claim 1, wherein the control unit is enclosed by the enclosing structure.

10. The massaging device as claimed in claim 9, wherein the irradiation characteristics comprise a mode of operation of the plurality of LEDs and frequencies of the irradiation.

11. The massaging device as claimed in claim 10, wherein the control unit comprises a communication interface configured to connect the massaging device with an external communication device and the control unit is configured to receive a control input from the external communication device.

12. The massaging device as claimed in claim 11, wherein the control input comprises a signal for modification of irradiation characteristics of the plurality of LEDs.

13. The massaging device as claimed in claim 1, wherein the enclosing structure is made up of a material selected from a group consisting of Acrylonitrile Butadiene Styrene (ABS), Polycarbonate (PC), natural stone, Himalayan stone, and combinations thereof.

14. The massaging device as claimed in claim 1, further comprising a vibratory motor configured to provide vibratory massage to the body of a user.

15. The massaging device as claimed in claim 1, further comprising a plurality of electrodes configured to provide Transcutaneous Electrical Nerve Stimulation (TENS), Electronic Muscle Stimulation (EMS), and Microcurrent Electrical Therapy (MET) to the body of a user.

16. The massaging device as claimed in claim 1, wherein enclosing structure is formed in shape of a Gua Sha stone.

17. A massaging device, the massaging device comprising:
an enclosing structure defining a cavity, the enclosing structure comprising a plurality of lateral portions, a top portion and a bottom portion;
a plurality of Light Emitting Diodes (LEDs) enclosed within the cavity;
a plurality of diaphanous portions, the plurality of diaphanous portions provided partially in the plurality of lateral portions, partially in the top portion, and partially in the bottom portion; and
wherein the plurality of diaphanous portions comprises a plurality of crest-shaped segments and a plurality of trough-shaped segments, and the plurality of LEDs are located in line with the plurality of trough-shaped segments.

18. A method of using a massaging device, the method comprising:
activating the massaging device using a user interface;
providing a control input to the massaging device from one or more of the user interface or an external computing device; and
applying a plurality of diaphanous portions to a face of a user.

19. The method as claimed in claim 18, further comprising activating a vibratory motor of the massaging device and providing a vibratory massage to the body of the user.

20. The method as claimed in claim 18, further comprising providing one or more of Transcutaneous Electrical Nerve Stimulation (TENS), Electronic Muscle Stimulation (EMS), and Microcurrent Electrical Therapy (MET) to a body of the user using a plurality of electrodes of the massaging device.
